SEQUENCE LISTING (50 sequences)

SEQ ID NO: 1 - protein, 12 aa, Artificial Sequence
  Synthetic Peptide Sequence
MASMTGGQQMGR

SEQ ID NO: 2 - protein, 23 aa, Artificial Sequence
  Synthetic Amino acid sequence
GSGQGQAQYLAASLVVFTNYSGD

SEQ ID NO: 3 - protein, 39 aa, Artificial Sequence
  Synthetic Amino acid sequence
MNNNDLFQASRRRFLAQLGGLTVAGMLGPSLLTPRRASA

SEQ ID NO: 4 - protein, 73 aa, Artificial Sequence
  Synthetic Amino acid sequence
MVLTKKKLQDLVREVAPNEQLDEDVEEMLLQIADDFIESVVTAACQLARHRKSSTLEVKDVQLHLERQWNMWI

SEQ ID NO: 5 - protein, 11 aa, Artificial Sequence
  Synthetic Amino acid sequence
SRRPRQLQQRQ

SEQ ID NO: 6 - protein, 22 aa, Artificial Sequence
  Synthetic Amino acid sequence
SEEPEQLQQEQSRRPRQLQQRQ

SEQ ID NO: 7 - protein, 31 aa, Artificial Sequence
  Synthetic Amino acid sequence
AEEEEILLEVSLVFKVKEFAPDAPLFTGPAY

SEQ ID NO: 8 - protein, 12 aa, Artificial Sequence
  Synthetic Amino acid sequence
SAGDLKFVKVVA

SEQ ID NO: 9 - protein, 13 aa, Artificial Sequence
  Synthetic Amino acid sequence
KTKQLMSFAPSHN

SEQ ID NO: 10 - protein, 125 aa, Artificial Sequence
  Synthetic Amino acid sequence
MHTPEHITAVVQRFVAALNAGDLDGIVALFADDATVEDPVGSEPRSGTAAIREFYANSLKLPLAVELTQEVRAVANEAAFAFTVSFEYQGRKTVVAPIDHFRFNGAGKVVSIRALFGEKNIHACQ

SEQ ID NO: 11 - protein, 6 aa, Artificial Sequence
  Synthetic Amino acid sequence
ENLYFQ

SEQ ID NO: 12 - protein, 31 aa, Artificial Sequence
  Synthetic Amino acid sequence
HAEGTFTSDVSSYLEGQAAKEFIAWLVRGRG

SEQ ID NO: 13 - protein, 55 aa, Artificial Sequence
  Synthetic Polypeptide sequence
MASMTGGQQMGRHHHHHHENLYFQHAEGTFTSDVSSYLEGQAAKEFIAWLVRGRG

SEQ ID NO: 14 - protein, 78 aa, Artificial Sequence
  Synthetic Polypeptide sequence
MASMTGGQQMGRHHHHHHGSGQGQAQYLAASLVVFTNYSGDENLYFQHAEGTFTSDVSSYLEGQAAKEFIAWLVRGRG

SEQ ID NO: 15 - protein, 94 aa, Artificial Sequence
  Synthetic Polypeptide sequence
MASMTGGQQMGRHHHHHHMNNNDLFQASRRRFLAQLGGLTVAGMLGPSLLTPRRASAENLYFQHAEGTFTSDVSSYLEGQAAKEFIAWLVRGRG

SEQ ID NO: 16 - protein, 128 aa, Artificial Sequence
  Synthetic Polypeptide sequence
MASMTGGQQMGRHHHHHHMVLTKKKLQDLVREVAPNEQLDEDVEEMLLQIADDFIESVVTAACQLARHRKSSTLEVKDVQLHLERQWNMWIENLYFQHAEGTFTSDVSSYLEGQAAKEFIAWLVRGRG

SEQ ID NO: 17 - protein, 66 aa, Artificial Sequence
  Synthetic Polypeptide sequence
MASMTGGQQMGRHHHHHHSRRPRQLQQRQENLYFQHAEGTFTSDVSSYLEGQAAKEFIAWLVRGRG

SEQ ID NO: 18 - protein, 77 aa, Artificial Sequence
  Synthetic Polypeptide sequence
MASMTGGQQMGRHHHHHHSEEPEQLQQEQSRRPRQLQQRQENLYFQHAEGTFTSDVSSYLEGQAAKEFIAWLVRGRG

SEQ ID NO: 19 - protein, 86 aa, Artificial Sequence
  Synthetic Polypeptide sequence
MASMTGGQQMGRHHHHHHAEEEEILLEVSLVFKVKEFAPDAPLFTGPAYENLYFQHAEGTFTSDVSSYLEGQAAKEFIAWLVRGRG

SEQ ID NO: 20 - protein, 67 aa, Artificial Sequence
  Synthetic Polypeptide sequence
MASMTGGQQMGRHHHHHHSAGDLKFVKVVAENLYFQHAEGTFTSDVSSYLEGQAAKEFIAWLVRGRG

SEQ ID NO: 21 - protein, 68 aa, Artificial Sequence
  Synthetic Polypeptide sequence
MASMTGGQQMGRHHHHHHKTKQLMSFAPSHNENLYFQHAEGTFTSDVSSYLEGQAAKEFIAWLVRGRG

SEQ ID NO: 22 - protein, 180 aa, Artificial Sequence
  Synthetic Polypeptide sequence
MASMTGGQQMGRHHHHHHMHTPEHITAVVQRFVAALNAGDLDGIVALFADDATVEDPVGSEPRSGTAAIREFYANSLKLPLAVELTQEVRAVANEAAFAFTVSFEYQGRKTVVAPIDHFRFNGAGKVVSIRALFGEKNIHACQENLYFQHAEGTFTSDVSSYLEGQAAKEFIAWLVRGRG

SEQ ID NO: 23 - protein, 55 aa, Artificial Sequence
  Synthetic Polypeptide sequence
MASMTGGQQMGRRRRRRRENLYFQHAEGTFTSDVSSYLEGQAAKEFIAWLVRGRG

SEQ ID NO: 24 - protein, 67 aa, Artificial Sequence
  Synthetic Polypeptide sequence
MHHHHHHGSGQGQAQYLAASLVVFTNYSGDENLYFQHAEGTFTSDVSSYLEGQAAKEFIAWLVRGRG

SEQ ID NO: 25 - protein, 56 aa, Artificial Sequence
  Synthetic Polypeptide sequence
MHHHHHHSAGDLKFVKVVAENLYFQHAEGTFTSDVSSYLEGQAAKEFIAWLVRGRG

SEQ ID NO: 26 - DNA, 69 bp, Artificial Sequence
  Synthetic Nucleic acid sequence
ggtagcggtcagggtcaagcacagtatctggcagcaagcctggttgtttttaccaattatagcggtgat

SEQ ID NO: 27 - DNA, 117 bp, Artificial Sequence
  Synthetic Nucleic acid sequence
atgaataacaacgacctgtttcaggcaagccgtcgtcgttttctggcacagttaggtggtctgaccgttgcaggtatgctgggtccgagcctgctgacaccgcgtcgtgcaagcgca

SEQ ID NO: 28 - DNA, 219 bp, Artificial Sequence
  Synthetic Nucleic acid sequence
atggttctgaccaaaaaaaagctgcaggatctggttcgtgaagttgcaccgaatgaacagctggatgaagatgttgaagaaatgctgctgcagattgccgatgattttattgaaagcgttgttaccgcagcatgtcagctggcacgtcatcgtaaaagcagcaccctggaagttaaagatgttcagctgcatctggaacgtcagtggaatatgtggatt

SEQ ID NO: 29 - DNA, 33 bp, Artificial Sequence
  Synthetic Nucleic acid sequence
agccgtcgtccgcgtcagctgcagcagcgtcaa

SEQ ID NO: 30 - DNA, 66 bp, Artificial Sequence
  Synthetic Nucleic acid sequence
agcgaagaaccggaacagctgcagcaagaacagagccgtcgtccgcgtcagctgcaacagcgtcaa

SEQ ID NO: 31 - DNA, 93 bp, Artificial Sequence
  Synthetic Nucleic acid sequence
gccgaagaagaagaaattctgctggaagttagcctggtgtttaaggtgaaagaatttgcaccggatgcaccgctgtttaccggtccggcatat

SEQ ID NO: 32 - DNA, 36 bp, Artificial Sequence
  Synthetic Nucleic acid sequence
tcagccggtgatctgaaatttgttaaagttgttgcc

SEQ ID NO: 33 - DNA, 39 bp, Artificial Sequence
  Synthetic Nucleic acid sequence
aaaaccaaacagctgatgagctttgcaccgagccataat

SEQ ID NO: 34 - DNA, 375 bp, Artificial Sequence
  Synthetic Nucleic acid sequence
atgcatacaccggaacatattaccgcagttgttcagcgttttgttgcagcactgaatgccggtgatctggatggtattgttgcactgtttgcagatgatgcaaccgttgaagatccggttggtagcgaaccgcgtagcggcaccgcagcaattcgtgaattttatgcaaatagcctgaaactgccgctggccgttgaactgacccaagaagttcgcgcagttgcaaatgaagcagcatttgcatttaccgtgagctttgaatatcagggtcgtaaaaccgttgttgcaccgattgatcattttcgttttaatggtgccggtaaagttgttagcattcgtgccctgtttggcgaaaaaaacattcatgcatgtcaa

SEQ ID NO: 35 - DNA, 168 bp, Artificial Sequence
  Synthetic Nucleic acid sequence
atggcaagcatgaccggtggtcagcagatgggtcgtcatcatcatcatcaccatgaaaacctgtattttcagcatgcagaaggcacctttacctcagatgttagcagctatctggaaggtcaggcagcaaaagaatttattgcatggctggttcgtggtcgtggttaa

SEQ ID NO: 36 - DNA, 237 bp, Artificial Sequence
  Synthetic Nucleic acid sequence
atggcaagcatgaccggtggtcagcagatgggtcgtcatcatcaccatcatcatggtagcggtcagggtcaagcacagtatctggcagcaagcctggttgtttttaccaattatagcggtgatgagaacctgtattttcagcatgcagaaggcacctttacctcagatgttagcagctatctggaaggtcaggcagcaaaagaatttattgcatggctggttcgtggtcgtggttaa

SEQ ID NO: 37 - DNA, 285 bp, Artificial Sequence
  Synthetic Nucleic acid sequence
atggcaagcatgaccggtggtcagcagatgggtcgtcatcatcaccatcatcatatgaataacaacgacctgtttcaggcaagccgtcgtcgttttctggcacagttaggtggtctgaccgttgcaggtatgctgggtccgagcctgctgacaccgcgtcgtgcaagcgcagaaaatctgtattttcagcatgcagaaggcacctttacctcagatgttagcagctatctggaaggtcaggcagcaaaagaatttattgcatggctggttcgtggtcgtggttaa

SEQ ID NO: 38 - DNA, 387 bp, Artificial Sequence
  Synthetic Nucleic acid sequence
atggcaagcatgaccggtggtcagcagatgggtcgtcatcatcaccatcatcatatggttctgaccaaaaaaaagctgcaggatctggttcgtgaagttgcaccgaatgaacagctggatgaagatgttgaagaaatgctgctgcagattgccgatgattttattgaaagcgttgttaccgcagcatgtcagctggcacgtcatcgtaaaagcagcaccctggaagttaaagatgttcagctgcatctggaacgtcagtggaatatgtggattgaaaacctgtattttcagcatgcagaaggcacctttacctcagatgttagcagttatctggaaggccaggcagcaaaagaatttattgcatggctggtgcgtggtcgtggttaa

SEQ ID NO: 39 - DNA, 201 bp, Artificial Sequence
  Synthetic Nucleic acid sequence
atggcaagcatgaccggtggtcagcagatgggtcgtcatcatcaccatcatcatagccgtcgtccgcgtcagctgcagcagcgtcaagaaaatctgtattttcagcatgcagaaggcacctttacctcagatgttagcagctatctggaaggtcaggcagcaaaagaatttattgcatggctggttcgtggtcgtggttaa

SEQ ID NO: 40 - DNA, 234 bp, Artificial Sequence
  Synthetic Nucleic acid sequence
atggcaagcatgaccggtggtcagcagatgggtcgtcatcatcaccatcatcatagcgaagaaccggaacagctgcagcaagaacagagccgtcgtccgcgtcagctgcaacagcgtcaagaaaatctgtattttcagcatgcagaaggcacctttacctcagatgttagcagctatctggaaggtcaggcagcaaaagaatttattgcatggctggttcgtggtcgtggttaa

SEQ ID NO: 41 - DNA, 261 bp, Artificial Sequence
  Synthetic Nucleic acid sequence
atggcaagcatgaccggtggtcagcagatgggtcgtcatcatcaccatcatcatgccgaagaagaagaaattctgctggaagttagcctggtgtttaaggtgaaagaatttgcaccggatgcaccgctgtttaccggtccggcatatgaaaatctgtattttcagcatgcagaaggcacctttacctcagatgttagcagctatctggaaggtcaggcagcaaaagaatttattgcatggctggttcgtggtcgtggttaa

SEQ ID NO: 42 - DNA, 204 bp, Artificial Sequence
  Synthetic Nucleic acid sequence
atggcaagcatgaccggtggtcagcagatgggtcgtcatcatcaccatcatcattcagccggtgatctgaaatttgttaaagttgttgccgagaacctgtattttcagcatgcagaaggcacctttacctcagatgttagcagctatctggaaggtcaggcagcaaaagaatttattgcatggctggttcgtggtcgtggttaa

SEQ ID NO: 43 - DNA, 207 bp, Artificial Sequence
  Synthetic Nucleic acid sequence
atggcaagcatgaccggtggtcagcagatgggtcgtcatcatcaccatcatcataaaaccaaacagctgatgagctttgcaccgagccataatgaaaatctgtattttcagcatgccgaaggcacctttaccagtgatgttagcagctatctggaaggtcaggcagcaaaagaatttattgcatggctggttcgtggtcgtggttaa

SEQ ID NO: 44 - DNA, 543 bp, Artificial Sequence
  Synthetic Nucleic acid sequence
atggcaagcatgaccggtggtcagcagatgggtcgtcatcatcaccatcatcatatgcatacaccggaacatattaccgcagttgttcagcgttttgttgcagcactgaatgccggtgatctggatggtattgttgcactgtttgcagatgatgcaaccgttgaagatccggttggtagcgaaccgcgtagcggcaccgcagcaattcgtgaattttatgcaaatagcctgaaactgccgctggccgttgaactgacccaagaagttcgcgcagttgcaaatgaagcagcatttgcatttaccgtgagctttgaatatcagggtcgtaaaaccgttgttgcaccgattgatcattttcgttttaatggtgccggtaaagttgttagcattcgtgccctgtttggcgaaaaaaacattcatgcatgtcaagaaaacctgtattttcagcatgcagaaggcacctttacctcagatgttagcagctatctggaaggtcaggcagcaaaagaatttattgcatggctggttcgtggtcgtggttaa

SEQ ID NO: 45 - DNA, 168 bp, Artificial Sequence
  Synthetic Nucleic acid sequence
atggcaagcatgaccggtggtcagcagatgggtcgtcgtcgccgtcgtcggcgtgaaaatctgtattttcagcatgcagaaggcacctttacctcagatgttagcagctatctggaaggtcaggcagcaaaagaatttattgcatggctggttcgtggtcgtggttaa

SEQ ID NO: 46 - DNA, 204 bp, Artificial Sequence
  Synthetic Nucleic acid sequence
atgcatcatcaccatcatcatggtagcggtcagggtcaagcacagtatctggcagcaagcctggttgtttttaccaattatagcggtgatgagaacctgtattttcagcatgcagaaggcacctttacctcagatgttagcagctatctggaaggtcaggcagcaaaagaatttattgcatggctggttcgtggtcgtggttaa

SEQ ID NO: 47 - DNA, 171 bp, Artificial Sequence
  Synthetic Nucleic acid sequence
atgcatcatcaccatcatcattcagccggtgatctgaaatttgttaaagttgttgccgagaacctgtattttcagcatgcagaaggcacctttacctcagatgttagcagctatctggaaggtcaggcagcaaaagaatttattgcatggctggttcgtggtcgtggttaa

SEQ ID NO: 48 - DNA, 93 bp, Artificial Sequence
  Synthetic Nucleic acid sequence
catgcagaaggcacctttacctcagatgttagcagctatctggaaggtcaggcagcaaaagaatttattgcatggctggttcgtggtcgtggt

SEQ ID NO: 49 - protein, 34 aa, Artificial Sequence
  Synthetic Amino acid sequence
SVSEIQLMHNLGKHLNSMERVEWLRKKLQDVHNF

SEQ ID NO: 50 - DNA, 102 bp, Artificial Sequence
  Synthetic Nucleic acid sequence
agcgttagcgaaattcagctgatgcataatctgggcaaacatctgaatagcatggaacgtgttgaatggctgcgtaaaaaactgcaggatgtgcacaacttt

(19) United States
(12) Patent Application Publication
(10) Pub. No.: US 2025/0059550 A1
(43) Pub. Date: Feb. 20, 2025

(54) CONSTRUCTS AND METHODS FOR INCREASED EXPRESSION OF POLYPEPTIDES

(71) Applicant: Biological E Limited, Hyderabad, Telangana (IN)

(72) Inventors: Pavan Reddy REGATTI, Hyderabad, Telangana (IN); Ramesh Venkat MATUR, Hyderabad, Telangana (IN); Narender Dev MANTENA, Hyderabad, Telangana (IN); Mahima DATLA, Hyderabad, Telangana (IN)

(21) Appl. No.: 18/553,006

(22) PCT Filed: Mar. 31, 2022
(86) PCT No.: PCT/IN2022/050327

§ 371 Date: Sep. 28, 2023

(30) Foreign Application Priority Data
Mar. 31, 2021 (IN) 202141014741

(51) Int. Cl. C12N 15/70; C07K 14/395; C07K 14/605; C07K 14/635

(52) U.S. Cl. CPC C12N 15/70; C07K 14/395; C07K 14/605; C07K 14/635; C07K 2319/21; C07K 2319/50

(57) ABSTRACT

The present invention relates to field of protein expression. It provides expression constructs and methods for increased expression of recombinant proteins. More particularly, it provides constructs and methods for enhanced expression of Lira-peptide in a recombinant host cell.

### CROSS REFERENCE TO RELATED APPLICATIONS

This This application is a 35 USC § 371 National Stage application of International Application No. PCT/IN2022/050327 entitled “CONSTRUCTS AND METHODS FOR INCREASED EXPRESSION OF POLYPEPTIDES,” filed on Mar. 31, 2022, which claims the benefit of priority to Indian Provisional Patent Application No. 202141014741, filed on 31 Mar. 2021, the entire contents of which are hereby incorporated by reference in their entireties.

### SEQUENCE LISTING

This application includes an electronically submitted sequence listing in .txt format. The .txt file contains a sequence listing entitled “117484.8018.US00_ST25.txt” created on Aug. 29, 2024 and is 26,334 bytes in size. The sequence listing contained in this .txt file is part of the specification and is hereby incorporated by reference herein in its entirety.

### FIELD OF INVENTION

The present invention relates to the field of protein expression. More specifically, it relates to constructs and methods for increased expression of recombinant polypeptides and proteins.

### BACKGROUND OF THE INVENTION

Peptide therapeutics have played a notable role in medical practice since the advent of insulin therapy in the 1920s. Currently, there are more than 60 approved peptide drugs in the market, and the numbers are expected to grow significantly.

Commercially useful proteins and peptides may be synthetically generated or isolated from natural sources. However, these methods are often expensive, time-consuming and characterized by limited production capacity. The preferred method of protein and peptide production is through the fermentation of recombinantly constructed organisms, engineered to overexpress the protein or peptide of interest.

However, recombinant expression of peptides has a number of obstacles to be overcome in order to be a cost-effective means of production. The obstacles are usually related to low expression levels of the recombinant protein or destruction of the expressed polypeptide by proteolytic enzymes contained within the cells.

Short peptides are challenging to produce recombinantly because they are susceptible to degradation in the cellular environment by host cellular proteases. Thus, the isolated product may be a heterogeneous mixture of species of the desired polypeptide having different amino acid chain lengths.

Additionally, purification can be difficult, resulting in poor yields depending on the nature of the protein or peptide of interest. The small peptides are being expressed by fusing to large fusion tags to overcome the above problem. Further, current methods use large fusion tags to express fusion proteins that decrease the potential yield of the peptide of interest. This is problematic in situations where the protein or peptide of interest is small in size.

It is advantageous to use small-size fusion tags to maximize the yield of the peptide of interest in such situations. But often, small tags rarely work as good as large tags.

These problems have been addressed in the past by producing fusion proteins that contain the desired polypeptide fused to a carrier polypeptide. Expression of the desired polypeptide as a fusion protein in a cell will often times protect the desired polypeptide from destructive enzymes and allow the fusion protein to be purified in high yields. The fusion protein is then treated to cleave the desired polypeptide from the carrier polypeptide and the desired polypeptide is isolated.

U.S. Pat. No. 7,572,884 discloses a method for preparing recombinant Lira-peptide, a precursor of Liraglutide in Saccharomyces cerevisiae.

U.S. Pat. No. 7,662,913 discloses the use of cystatin-based peptide tags, which is used for generating insoluble fusion peptides.

U.S. Pat. No. 8,796,431 discloses methods and processes for the efficient production of peptides including GLP1 using keto-steroid isomerase (KSI) as inclusion body partner.

WO 2003/100021 A1 discloses expression cassette for increased production of a heterologous peptides/proteins comprising of promoter, translation initiation sequence, inclusion body fusion partner and a cleavable linker operably linked to the heterologous protein.

WO 2017/021819 A1 discloses a process for the preparation of peptides or proteins or derivatives thereof by expression of synthetic oligonucleotide encoding desired protein or peptide in a prokaryotic cell as ubiquitin fusion construct.

IN 201741024763 A discloses a process for the preparation of Liraglutide by expression of synthetic oligonucleotide encoding Lira-peptide which is operably connected to an oligonucleotide sequence of a signal peptide in a yeast cell.

Yang Liu et al., (Biotechnol Lett 36, 1675-1680 (2014)) explains a strategy for expression and purification of functional GLP-1 peptide using glutathione S-transferase (GST) fusion tag of 23 kDa, with an enterokinase cleavage site in the fusion junction in E. coli.

Zhao et al., (Microb Cell Fact 15, 136 (2016)) Studies recombinant expression of a cleavable self-aggregating tag and intein-mediated cleavage of medium to large-sized peptides including GLP1 in Escherichia coli.

Zhao et al., (Microb Cell Fact 18, 91 (2019)) studies the use of Self-assembling amphipathic peptides (SAPs) as expression tag to enhance recombinant enzyme production.

Ki et al., (Appl Microbiol Biotechnol. 2020 March; 104 (6): 2411-2425) provides a detailed review of fusion tags that increase the expression of heterologous proteins in E. coli.

Glucagon-like peptide-1 (GLP-1) is a 31 amino acid long peptide hormone deriving from the tissue-specific post-translational processing of the proglucagon peptide. It is produced and secreted by intestinal enteroendocrine L-cells and certain neurons within the nucleus of the solitary tract in the brainstem upon food consumption. Liraglutide is a derivative of a human incretin (metabolic hormone), glucagon-like peptide-1 (GLP-1) that is used as a long-acting glucagon-like peptide-1 receptor agonist, binding to the same receptors as the endogenous metabolic hormone

GLP-1 that stimulates insulin secretion. Accordingly, there exists a need for novel expression strategies to increase the expression of recombinant proteins in the host. The inventors of the present invention, in their endeavor to enhance the expression of the recombinant therapeutic peptides by several folds, have come up with expression constructs, which allow high yield production of recombinant proteins.

### OBJECTIVE OF THE INVENTION

It is the main objective of the invention to provide an expression cassette for producing a protein of interest with high yield.

Another objective of the invention to provide a method for increased expression of a protein of interest.

### SUMMARY OF THE INVENTION

The present invention provides expression constructs, vectors and recombinant host cells for increased expression and efficient production of biologically active peptides such as lira-peptide.

In an embodiment, the present invention provides an expression cassette for expression of a protein of interest comprising:
a) polynucleotide encoding T7 leader polypeptide comprising the amino acid sequence of SEQ ID NO: 1;
b) a polynucleotide encoding expression tag polypeptide comprising of an amino acid sequence selected from the group comprising of SEQ ID No: 2-10;
c) polynucleotide encoding a cleavable peptide linker; and
d) a polynucleotide encoding the protein of interest,
   wherein the said polynucleotide sequences of the expression cassette are operably linked to a promoter.

In a particular embodiment, the invention provides a fusion polypeptide comprising of:
a) a T7 leader polypeptide comprising the amino acid sequence of SEQ ID NO: 1;
b) an expression tag polypeptide having an amino acid sequence selected from group comprising of SEQ ID NOs: 2-10; and
c) a cleavable peptide linker;
fused to the amino-terminal of a protein of interest to obtain the fusion polypeptide.

The present invention provides an expression cassette for expression of lira-peptide comprising:
a) polynucleotide encoding T7 leader polypeptide comprising the amino acid sequence of SEQ ID NO: 1;
b) a polynucleotide encoding expression tag polypeptide comprising of an amino acid sequence selected from the group comprising of SEQ ID No: 2-10;
c) a polynucleotide encoding a cleavable linker; and
d) a polynucleotide encoding lira-peptide comprising the amino acid sequence as set forth in SEQ ID No: 12 or functional variant thereof,
   wherein the said polynucleotide sequences of the expression cassette are operably linked to a promoter.

In an embodiment, the invention provides a fusion polypeptide comprising of:
a) a T7 leader polypeptide comprising the amino acid sequence of SEQ ID NO: 1;
b) an expression tag polypeptide having an amino acid sequence selected from group comprising of SEQ ID NOs: 2-10; and
c) a cleavable linker peptide;
fused to the amino terminal of lira-peptide comprising the amino acid sequence of SEQ ID NO: 12 or functional variant thereof, to obtain the fusion polypeptide.

In an embodiment of the present invention, the expression level of the protein of interest increases by at least 85%.

### BRIEF DESCRIPTION OF DRAWINGS:

FIG. 1A: Schematic diagram of Expression cassette without N-terminal expression tag fusion

FIG. 1B: Schematic diagram of the expression cassette(s) with the N-terminal expression tags (LP2 to LP10) along with T7 leader.

FIG. 1C: Schematic diagram of the expression cassette with the N-terminal expression tag (LP2) without T7 leader.

FIG. 1D: Schematic diagram of the expression cassette with the N-terminal expression tag (LP8) without T7 leader.

FIG. 2A: Schematic diagram of the expression vector LP1 (without any N-terminal expression tag)

FIG. 2B: Schematic diagram of the expression vector with the T7 leader and N-terminal expression tag (LP-2).

FIG. 2C: Schematic diagram of the expression vector without T7 leader and with the N-terminal expression tag (LP-2).

FIG. 2D: Schematic diagram of the expression vector with T7 leader and N-terminal expression tag (LP-8).

FIG. 2E: Schematic diagram of the expression vector without T7 leader and with the N-terminal expression tag (LP-8).

FIG. 3A: Clones, with different expression tag sequence, tested for expression of Lira-peptide.

FIG. 3B: The table represents the molecular weight of each cassette and percentage of tagged lira-peptide per lane, based on densitometry analysis.

FIG. 4A: Comparative lira-peptide expression in presence and absence of T7 leader sequence with LP-2 expression tag in the expression cassette.

FIG. 4 B: Densitometry analysis for lira-peptide expression in the presence and absence of T7leader sequence with LP-2 expression tag in the expression cassette.

FIG. 4C: Percentage increase in expression of lira-peptide (LP2) with T7 leader when compared to without T7-leader

FIG. 5A: Comparative lira-peptide expression in presence and absence of T7 leader sequence with LP-8 expression tag in the expression cassette.

FIG. 5B: Densitometry analysis for lira-peptide expression in the presence and absence of T7leader sequence with LP-8 expression tag in the expression cassette.

FIG. 5C: Percentage increase in expression of lira-peptide (LP8) with T7 leader when compared to without T7-leader

FIG. 6A: Purification of Lira-peptide containing N-terminal fusion using Ni-NTA chromatography

FIG. 6B: Purification of Lira-peptide using reverse phase chromatography

FIG. 7: Lira-peptide expression in soluble and insoluble fractions.

FIG. 8: Clones, with different expression tag sequence, tested for expression of Teriparatide.

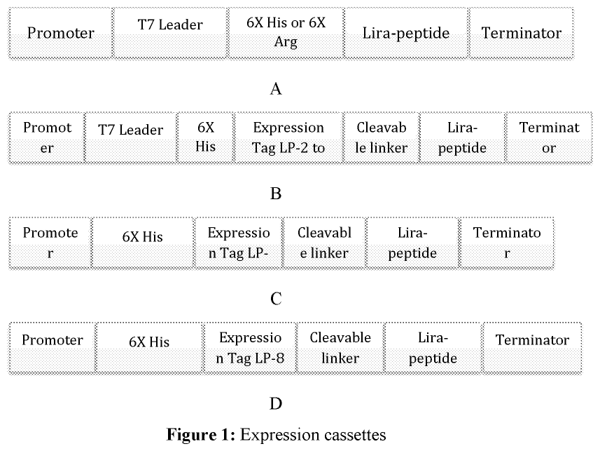

### DESCRIPTION OF SEQUENCE LISTING

(T7 leader sequence)
SEQ ID NO: 1
MASMTGGQQMGR
(amino acid sequence of expression tag LP-2)
SEQ ID NO: 2
GSGQGQAQYLAASLVVFTNYSGD
(amino acid sequence of expression tag LP-3
SEQ ID NO: 3
MNNNDLFQASRRRFLAQLGGLTVAGMLGPSLLTPRRASA
(amino acid sequence of expression tag LP-4)
SEQ ID NO: 4
MVLTKKKLQDLVREVAPNEQLDEDVEEMLLQIADDFIESVVTAACQLARHRKSSTLEV
KDVQLHLERQWNMWI
(amino acid sequence of expression tag LP-5)
SEQ ID NO: 5
SRRPRQLQQRQ
(amino acid sequence of expression tag LP-6)
SEQ ID NO: 6
SEEPEQLQQEQSRRPRQLQQRQ
(amino acid sequence of expression tag LP-7)
SEQ ID NO: 7
AEEEEILLEVSLVFKVKEFAPDAPLFTGPAY
(amino acid sequence of expression tag LP-8)
SEQ ID NO: 8
SAGDLKFVKVVA
(amino acid sequence of expression tag LP-9)
SEQ ID NO: 9
KTKQLMSFAPSHN
(amino acid sequence of expression tag LP-10)
SEQ ID NO: 10
MHTPEHITAVVQRFVAALNAGDLDGIVALFADDATVEDPVGSEPRSGTAAIREFYANSL
KLPLAVELTQEVRAVANEAAFAFTVSFEYQGRKTVVAPIDHFRFNGAGKVVSIRALFGE
KNIHACQ
(amino acid sequence of the TEV cleavage site)
SEQ ID NO: 11
ENLYFQ
(amino acid sequence of Lira-peptide)
SEQ ID NO: 12
HAEGTFTSDVSSYLEGQAAKEFIAWLVRGRG
(expression cassette LP1) consists of T7 leader + 6XHis +
TEVrecognition site + Lira-peptide)
SEQ ID NO: 13
MASMTGGQQMGRHHHHHHENLYFQHAEGTFTSDVSSYLEGQAAKEFIAWLVRGRG
(expression cassette LP2) consists of T7 leader + 6XHis +
expression tag LP2 + TEVrecognition site + Lira-peptide)
SEQ ID NO: 14
MASMTGGQQMGRHHHHHHGSGQGQAQYLAASLVVFTNYSGDENLYFQHAEGTFTSD
VSSYLEGQAAKEFIAWLVRGRG
(expression cassette LP3) consists of T7 leader + 6XHis +
expression tag LP3 + TEVrecognition site + Lira-peptide)
SEQ ID NO: 15
MASMTGGQQMGRHHHHHHMNNNDLFQASRRRFLAQLGGLTVAGMLGPSLLTPRRAS
AENLYFQHAEGTFTSDVSSYLEGQAAKEFIAWLVRGRG
(expression cassette LP4) consists of T7 leader +
6XHis + expression tag LP4 + TEVrecognition site + Lira-peptide)
SEQ ID NO: 16
MASMTGGQQMGRHHHHHHMVLTKKKLQDLVREVAPNEQLDEDVEEMLLQIADDFIES
VVTAACQLARHRKSSTLEVKDVQLHLERQWNMWIENLYFQHAEGTFTSDVSSYLEGQ
AAKEFIAWLVRGRG
(expression cassette LP5) consists of T7 leader + 6XHis +
expression tag LP5 +T EVrecognition site + Lira-peptide)
SEQ ID NO: 17
MASMTGGQQMGRHHHHHHSRRPRQLQQRQENLYFQHAEGTFTSDVSSYLEGQAAKEF
IAWLVRGRG
(expression cassette LP6) consists of T7 leader +
6XHis + expression tag LP6 + TEVrecognition site + Lira-peptide)
SEQ ID NO: 18
MASMTGGQQMGRHHHHHHSEEPEQLQQEQSRRPRQLQQRQENLYFQHAEGTFTSDVS
SYLEGQAAKEFIAWLVRGRG
(expression cassette LP7) consists of T7 leader + 6XHis +
expression tag LP7 + TEVrecognition site + Lira-peptide)
SEQ ID NO: 19
MASMTGGQQMGRHHHHHHAEEEEILLEVSLVFKVKEFAPDAPLFTGPAYENLYFQHAE
GTFTSDVSSYLEGQAAKEFIAWLVRGRG
(expression cassette LP8) consists of T7 leader +
6XHis + expression tag LP8 + TEVrecognition site + Lira-peptide)
SEQ ID NO: 20
MASMTGGQQMGRHHHHHHSAGDLKFVKVVAENLYFQHAEGTFTSDVSSYLEGQAAK
EFIAWLVRGRG
(expression cassette LP9) consists of T7 leader + 6XHis +
expression tag LP9 + TEVrecognition site + Lira-peptide)
SEQ ID NO: 21
MASMTGGQQMGRHHHHHHKTKQLMSFAPSHNENLYFQHAEGTFTSDVSSYLEGQAA
KEFIAWLVRGRG
(expression cassette LP10) consists of T7 leader + 6XHis +
expression tag LP10 + TEVrecognition site + Lira-peptide)
SEQ ID NO: 22
MASMTGGQQMGRHHHHHHMHTPEHITAVVQRFVAALNAGDLDGIVALFADDATVED
PVGSEPRSGTAAIREFYANSLKLPLAVELTQEVRAVANEAAFAFTVSFEYQGRKTVVAPI
DHFRFNGAGKVVSIRALFGEKNIHACQENLYFQHAEGTFTSDVSSYLEGQAAKEFIAWL
VRGRG
(expression cassette LP11) consists of T7 leader + 6XArg +
TEVrecognition site + Lira-peptide)
SEQ ID NO: 23
MASMTGGQQMGRRRRRRRENLYFQHAEGTFTSDVSSYLEGQAAKEFIAWLVRGRG
(expression cassette LP2 without T7 leader) consists of 6XHis +
expression tag LP2 + TEVrecognition site + Lira-peptide)
SEQ ID NO: 24
MHHHHHHGSGQGQAQYLAASLVVFTNYSGDENLYFQHAEGTFTSDVSSYLEGQAAKE
FIAWLVRGRG
(expression cassette LP8 without T7 leader) consists of 6XHis +
expression tag LP8 + TEVrecognition site + Lira-peptide)
SEQ ID NO: 25
MHHHHHHSAGDLKFVKVVAENLYFQHAEGTFTSDVSSYLEGQAAKEFIAWLVRGRG
(Nucleic acid sequence encoding SEQ ID NO: 2-expression tag LP-2)
SEQ ID NO: 26
GGTAGCGGTCAGGGTCAAGCACAGTATCTGGCAGCAAGCCTGGTTGTTTTTACCAAT
TATAGCGGTGAT
(nucleic acid sequence encoding SEQ ID NO: 3-expression tag LP-3)
SEQ ID NO: 27
ATGAATAACAACGACCTGTTTCAGGCAAGCCGTCGTCGTTTTCTGGCACAGTTAGGT
GGTCTGACCGTTGCAGGTATGCTGGGTCCGAGCCTGCTGACACCGCGTCGTGCAAGC
GCA
(nucleic acid sequence encoding SEQ ID NO: 4-expression tag LP-4)
SEQ ID NO: 28
ATGGTTCTGACCAAAAAAAAGCTGCAGGATCTGGTTCGTGAAGTTGCACCGAATGA
ACAGCTGGATGAAGATGTTGAAGAAATGCTGCTGCAGATTGCCGATGATTTTATTGA
AAGCGTTGTTACCGCAGCATGTCAGCTGGCACGTCATCGTAAAAGCAGCACCCTGG
AAGTTAAAGATGTTCAGCTGCATCTGGAACGTCAGTGGAATATGTGGATT
(nucleic acid sequence encoding SEQ ID NO: 5-expression tag LP-5)
SEQ ID NO: 29
AGCCGTCGTCCGCGTCAGCTGCAGCAGCGTCAA
(nucleic acid sequence encoding SEQ ID NO: 6-expression tag LP-6)
SEQ ID NO: 30
AGCGAAGAACCGGAACAGCTGCAGCAAGAACAGAGCCGTCGTCCGCGTCAGCTGCA
ACAGCGTCAA
(nucleic acid sequence encoding SEQ ID NO: 7-expression tag LP-7)
SEQ ID NO: 31
GCCGAAGAAGAAGAAATTCTGCTGGAAGTTAGCCTGGTGTTTAAGGTGAAAGAATT
TGCACCGGATGCACCGCTGTTTACCGGTCCGGCATAT
(nucleic acid sequence encoding SEQ ID NO: 8-expression tag LP-8)
SEQ ID NO: 32
TCAGCCGGTGATCTGAAATTTGTTAAAGTTGTTGCC
(nucleic acid sequence encoding SEQ ID NO: 9-expression tag LP-9)
SEQ ID NO: 33
AAAACCAAACAGCTGATGAGCTTTGCACCGAGCCATAAT
(nucleic acid sequence encoding SEQ ID NO: 10-expression tag LP-10)
SEQ ID NO: 34
ATGCATACACCGGAACATATTACCGCAGTTGTTCAGCGTTTTGTTGCAGCACTGAAT
GCCGGTGATCTGGATGGTATTGTTGCACTGTTTGCAGATGATGCAACCGTTGAAGAT
CCGGTTGGTAGCGAACCGCGTAGCGGCACCGCAGCAATTCGTGAATTTTATGCAAAT
AGCCTGAAACTGCCGCTGGCCGTTGAACTGACCCAAGAAGTTCGCGCAGTTGCAAA
TGAAGCAGCATTTGCATTTACCGTGAGCTTTGAATATCAGGGTCGTAAAACCGTTGT
TGCACCGATTGATCATTTTCGTTTTAATGGTGCCGGTAAAGTTGTTAGCATTCGTGCC
CTGTTTGGCGAAAAAAACATTCATGCATGTCAA
(expression cassette encoding SEQ ID NO: 13-LP1 nucleic acid
sequence consisting of T7 leader + 6XHis + TEVrecognition site +
Lira-peptide)
SEQ ID NO: 35
ATGGCAAGCATGACCGGTGGTCAGCAGATGGGTCGTCATCATCATCATCACCATGA
AAACCTGTATTTTCAGCATGCAGAAGGCACCTTTACCTCAGATGTTAGCAGCTATCT
GGAAGGTCAGGCAGCAAAAGAATTTATTGCATGGCTGGTTCGTGGTCGTGGTTAA
(expression cassette encoding SEQ ID NO: 14-LP2 nucleic acid
sequence consisting of T7 leader + 6XHis + expression tag LP2 +
TEVrecognition site + Lira-peptide)
SEQ ID NO: 36
ATGGCAAGCATGACCGGTGGTCAGCAGATGGGTCGTCATCATCACCATCATCATGGT
AGCGGTCAGGGTCAAGCACAGTATCTGGCAGCAAGCCTGGTTGTTTTTACCAATTAT
AGCGGTGATGAGAACCTGTATTTTCAGCATGCAGAAGGCACCTTTACCTCAGATGTT
AGCAGCTATCTGGAAGGTCAGGCAGCAAAAGAATTTATTGCATGGCTGGTTCGTGGT
CGTGGTTAA
(expression cassette encoding SEQ ID NO: 15-LP3 nucleic acid
sequence consisting of T7 leader + 6XHis + expression tag LP3 +
TEVrecognition site + Lira-peptide)
SEQ ID NO: 37
ATGGCAAGCATGACCGGTGGTCAGCAGATGGGTCGTCATCATCACCATCATCATATG
AATAACAACGACCTGTTTCAGGCAAGCCGTCGTCGTTTTCTGGCACAGTTAGGTGGT
CTGACCGTTGCAGGTATGCTGGGTCCGAGCCTGCTGACACCGCGTCGTGCAAGCGCA
GAAAATCTGTATTTTCAGCATGCAGAAGGCACCTTTACCTCAGATGTTAGCAGCTAT
CTGGAAGGTCAGGCAGCAAAAGAATTTATTGCATGGCTGGTTCGTGGTCGTGGTTAA
(expression cassette encoding SEQ ID NO: 16-LP4 nucleic acid
sequence consisting of T7 leader + 6XHis + expression tag LP4 +
TEVrecognition site + Lira-peptide)
SEQ ID NO: 38
ATGGCAAGCATGACCGGTGGTCAGCAGATGGGTCGTCATCATCACCATCATCATATG
GTTCTGACCAAAAAAAAGCTGCAGGATCTGGTTCGTGAAGTTGCACCGAATGAACA
GCTGGATGAAGATGTTGAAGAAATGCTGCTGCAGATTGCCGATGATTTTATTGAAAG
CGTTGTTACCGCAGCATGTCAGCTGGCACGTCATCGTAAAAGCAGCACCCTGGAAGT
TAAAGATGTTCAGCTGCATCTGGAACGTCAGTGGAATATGTGGATTGAAAACCTGTA
TTTTCAGCATGCAGAAGGCACCTTTACCTCAGATGTTAGCAGTTATCTGGAAGGCCA
GGCAGCAAAAGAATTTATTGCATGGCTGGTGCGTGGTCGTGGTTAA
(expression cassette encoding SEQ ID NO: 17-LP5 nucleic acid
sequence consisting of T7 leader + 6XHis + expression tag LP5 +
TEVrecognition site + Lira-peptide)
SEQ ID NO: 39
ATGGCAAGCATGACCGGTGGTCAGCAGATGGGTCGTCATCATCACCATCATCATAGC
CGTCGTCCGCGTCAGCTGCAGCAGCGTCAAGAAAATCTGTATTTTCAGCATGCAGAA
GGCACCTTTACCTCAGATGTTAGCAGCTATCTGGAAGGTCAGGCAGCAAAAGAATTT
ATTGCATGGCTGGTTCGTGGTCGTGGTTAA
(expression cassette encoding SEQ ID NO: 18-LP6 nucleic acid
sequence consisting of T7 leader + 6XHis + expression tag LP6 +
TEVrecognition site + Lira-peptide)
SEQ ID NO: 40
ATGGCAAGCATGACCGGTGGTCAGCAGATGGGTCGTCATCATCACCATCATCATAGC
GAAGAACCGGAACAGCTGCAGCAAGAACAGAGCCGTCGTCCGCGTCAGCTGCAACA
GCGTCAAGAAAATCTGTATTTTCAGCATGCAGAAGGCACCTTTACCTCAGATGTTAG
CAGCTATCTGGAAGGTCAGGCAGCAAAAGAATTTATTGCATGGCTGGTTCGTGGTCG
TGGTTAA
(expression cassette encoding SEQ ID NO: 19-LP7 nucleic acid
sequence consisting of T7 leader + 6XHis + expression tag LP7 +
TEVrecognition site + Lira-peptide)
SEQ ID NO: 41
ATGGCAAGCATGACCGGTGGTCAGCAGATGGGTCGTCATCATCACCATCATCATGCC
GAAGAAGAAGAAATTCTGCTGGAAGTTAGCCTGGTGTTTAAGGTGAAAGAATTTGC
ACCGGATGCACCGCTGTTTACCGGTCCGGCATATGAAAATCTGTATTTTCAGCATGC
AGAAGGCACCTTTACCTCAGATGTTAGCAGCTATCTGGAAGGTCAGGCAGCAAAAG
AATTTATTGCATGGCTGGTTCGTGGTCGTGGTTAA
(expression cassette encoding SEQ ID NO: 20-LP8 nucleic acid
sequence consisting of T7 leader + 6XHis + expression tag LP8 +
TEVrecognition site + Lira-peptide)
SEQ ID NO: 42
ATGGCAAGCATGACCGGTGGTCAGCAGATGGGTCGTCATCATCACCATCATCATTCA
GCCGGTGATCTGAAATTTGTTAAAGTTGTTGCCGAGAACCTGTATTTTCAGCATGCA
GAAGGCACCTTTACCTCAGATGTTAGCAGCTATCTGGAAGGTCAGGCAGCAAAAGA
ATTTATTGCATGGCTGGTTCGTGGTCGTGGTTAA
(expression cassette encoding SEQ ID NO: 21-LP9 nucleic acid
sequence consisting of T7 leader + 6XHis + expression tag LP9 +
TEVrecognition site + Lira-peptide)
SEQ ID NO: 43
ATGGCAAGCATGACCGGTGGTCAGCAGATGGGTCGTCATCATCACCATCATCATAA
AACCAAACAGCTGATGAGCTTTGCACCGAGCCATAATGAAAATCTGTATTTTCAGCA
TGCCGAAGGCACCTTTACCAGTGATGTTAGCAGCTATCTGGAAGGTCAGGCAGCAA
AAGAATTTATTGCATGGCTGGTTCGTGGTCGTGGTTAA
(expression cassette encoding SEQ ID NO: 22-LP10 nucleic acid
sequence) consists of T7 leader + 6XHis + expression tag LP10 +
TEVrecognition site + Lira-peptide)
SEQ ID NO: 44
ATGGCAAGCATGACCGGTGGTCAGCAGATGGGTCGTCATCATCACCATCATCATATG
CATACACCGGAACATATTACCGCAGTTGTTCAGCGTTTTGTTGCAGCACTGAATGCC
GGTGATCTGGATGGTATTGTTGCACTGTTTGCAGATGATGCAACCGTTGAAGATCCG
GTTGGTAGCGAACCGCGTAGCGGCACCGCAGCAATTCGTGAATTTTATGCAAATAGC
CTGAAACTGCCGCTGGCCGTTGAACTGACCCAAGAAGTTCGCGCAGTTGCAAATGA
AGCAGCATTTGCATTTACCGTGAGCTTTGAATATCAGGGTCGTAAAACCGTTGTTGC
ACCGATTGATCATTTTCGTTTTAATGGTGCCGGTAAAGTIGTTAGCATTCGTGCCCTG
TTTGGCGAAAAAAACATTCATGCATGTCAAGAAAACCTGTATTTTCAGCATGCAGAA
GGCACCTTTACCTCAGATGTTAGCAGCTATCTGGAAGGTCAGGCAGCAAAAGAATTT
ATTGCATGGCTGGTTCGTGGTCGTGGTTAA
(expression cassette encoding SEQ ID NO: 23-LP11 nucleic acid
sequence consisting of T7 leader + 6XArg + TEVrecognition site +
Lira-peptide)
SEQ ID NO: 45
ATGGCAAGCATGACCGGTGGTCAGCAGATGGGTCGTCGTCGCCGTCGTCGGCGTGA
AAATCTGTATTTTCAGCATGCAGAAGGCACCTTTACCTCAGATGTTAGCAGCTATCT
GGAAGGTCAGGCAGCAAAAGAATTTATTGCATGGCTGGTTCGTGGTCGTGGTTAA
(expression cassette encoding SEQ ID NO: 24-LP2 without T7 leader
nucleic acid sequence consisting of 6XHis + expression tag LP2 +
TEVrecognition site + Lira-peptide)
SEQ ID NO: 46
ATGCATCATCACCATCATCATGGTAGCGGTCAGGGTCAAGCACAGTATCTGGCAGCA
AGCCTGGTTGTTTTTACCAATTATAGCGGTGATGAGAACCTGTATTTTCAGCATGCA
GAAGGCACCTTTACCTCAGATGTTAGCAGCTATCTGGAAGGTCAGGCAGCAAAAGA
ATTTATTGCATGGCTGGTTCGTGGTCGTGGTTAA
(expression cassette encoding SEQ ID NO: 25-LP8 without T7 leader
nucleic acid sequence consisting of 6XHis + expression tag LP8 +
TEVrecognition site + Lira-peptide)
SEQ ID NO: 47
ATGCATCATCACCATCATCATTCAGCCGGTGATCTGAAATTTGTTAAAGTTGTTGCC
GAGAACCTGTATTTTCAGCATGCAGAAGGCACCTTTACCTCAGATGTTAGCAGCTAT
CTGGAAGGTCAGGCAGCAAAAGAATTTATTGCATGGCTGGTTCGTGGTCGTGGTTAA
(Codon optimized nucleic acid sequence encoding Lira-peptide)
SEQ ID NO: 48
CATGCAGAAGGCACCTTTACCTCAGATGTTAGCAGCTATCTGGAAGGTCAGGCAGC
AAAAGAATTTATTGCATGGCTGGTTCGTGGTCGTGGT
(amino acid sequence of Teriparatide)
SEQ ID NO: 49
SVSEIQLMHNLGKHLNSMERVEWLRKKLQDVHNF
(Codon optimized nucleic acid sequence encoding Teriparatide)
SEQ ID NO: 50
AGCGTTAGCGAAATTCAGCTGATGCATAATCTGGGCAAACATCTGAATAGCATGGA
ACGTGTTGAATGGCTGCGTAAAAAACTGCAGGATGTGCACAACTTT

### Definitions

Unless defined otherwise, all technical and scientific terms used herein have the same meaning as commonly understood by one of ordinary skill in the art to which the methods belong. Although any vectors, host cells, methods, and compositions similar or equivalent to those described herein can also be used in the practice or testing of the vectors, host cells, methods, and compositions, representative illustrations are now described.

Where a range of values is provided, it is understood that each intervening value between the upper and lower limit of that range and any other stated or intervening value in that stated range, is encompassed within by the methods and compositions. The upper and lower limits of these smaller ranges may independently be included in the smaller ranges and are also encompassed within by the methods and compositions, subject to any specifically excluded limit in the stated range. Where the stated range includes one or both of the limits, ranges excluding either or both of those included limits are also included in the methods and compositions.

It is appreciated that certain features of the methods, which are, for clarity, described in the context of separate embodiments, may also be provided in combination in a single embodiment. Conversely, various features of the methods and compositions, which are, for brevity, described in the context of a single embodiment, may also be provided separately or in any suitable sub-combination. It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

As will be apparent to those of skill in the art upon reading this disclosure, each of the individual embodiments described and illustrated herein has discrete components and features which may be readily separated from or combined with the features of any of the other embodiments without departing from the scope or spirit of the present methods. Any recited method can be carried out in the order of events recited or in any other order that is logically possible.

The term “host cell” includes an individual cell or cell culture which can be, or has been, a recipient for the subject of expression constructs. Host cells include the progeny of a single host cell. Preferable host cell is Escherichia coli, also known as E. coli, which is a Gram-negative, facultative anaerobic, rod-shaped, coliform bacterium of the genus Escherichia that is commonly found in the lower intestine of warm-blooded organisms and Corynebacterium glutamicum and Bacillus subtilis.

The term “recombinant strain” or “recombinant host cell” refers to a host cell which has been transfected or transformed with the expression constructs or vectors of this invention.

The term “expression” refers to the biological production of a product encoded by a coding sequence. In most cases, a DNA sequence, including the coding sequence, is transcribed to form a messenger-RNA (mRNA). The messenger-RNA is then translated to form a polypeptide product that has a relevant biological activity. Also, the process of expression may involve further processing steps to the RNA product of transcription, such as splicing to remove introns, and/or post-translational processing of a polypeptide product.

The term “expression vector” or “expression construct” refers to any vector, plasmid or vehicle designed to enable the expression of an inserted nucleic acid sequence following transformation into the host.

The term “cassette” or “expression cassette” refer to a segment of DNA that can be inserted into a nucleic acid or polynucleotide at specific restriction sites. The segment of DNA comprises a polynucleotide that encodes a protein of interest. “cassette” or “expression cassette” may also comprise elements that allow for enhanced expression of a polynucleotide encoding a protein of interest in a host cell These elements may include, but are not limited to: a promoter, an enhancer a response element, a terminator sequence, a polyadenylation sequence, and the like.

The term “promoter” refers to a DNA sequences that define where transcription of a gene begins. Promoter sequences are typically located directly upstream or at the 540 end of the transcription initiation site. RNA polymerase and the necessary transcription factors bind to the promoter sequence and initiate transcription. Promoters can either be constitutive or inducible promoters. Constitutive promoters are the promoter which allows continual transcription of its associated genes as their expression is normally not conditioned by environmental and developmental factors. Constitutive promoters are very useful tools in genetic engineering because constitutive promoters drive gene expression under inducer-free conditions and often show better characteristics than commonly used inducible promoters. Inducible promoters are the promoters that are induced by the presence or absence of biotic or abiotic and chemical or physical factors. Inducible promoters are a very powerful tool in genetic engineering because the expression of genes operably linked to them can be turned on or off at certain stages of development or growth of an organism or in a particular tissue or cells.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter).

The term “expression tag” as used herein refers to any peptide or polypeptide that can be attached to a protein of interest and is supposed to support the solubility, stability and/or the expression of a recombinant protein of interest.

A “Cleavable linker peptide” refers to a peptide sequence having a cleavage recognition sequence. A cleavable peptide linker can be cleaved by an enzymatic or a chemical cleavage agent.

The terms “polypeptide”, “peptide” and “protein” are used interchangeably herein to refer to two or more amino acid residues joined to each other by peptide bonds or modified peptide bonds. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer. “Polypeptide” refers to both short chains, commonly referred to as peptides, oligopeptides or oligomers, and to longer chains, generally referred to as proteins. Polypeptides may contain amino acids other than the 20 gene-encoded amino acids. Likewise, “protein” refers to at least two covalently attached amino acids, which includes proteins, polypeptides, oligopeptides and peptides. A protein may be made up of naturally occurring amino acids and peptide bonds, or synthetic peptidomimetic structures. Thus “amino acid”, or “peptide residue”, as used herein means both naturally occurring and synthetic amino acids. “Amino acid” includes imino acid residues such as proline and hydroxyproline. The side chains may be in either the (R) or the(S) configuration.

### DETAILED DESCRIPTION OF THE INVENTION

The present invention provides expression constructs, vectors and recombinant host cells for increased expression and efficient production of biologically active peptides such as lira-peptide.

Peptides produced according to the invention may be produced more efficiently than peptides produced according to prior art processes, because of using the short fusion tags. Current methods use large fusion tags for the expression of fusion proteins that decrease the potential yield of desired peptide of interest. This is particularly problematic in situations where the desired peptide is small like lira-peptide which is 31 amino acids. In such situations it is advantageous to use a smallest possible fusion tag to maximized yield.

The invention contemplates a multidimensional approach for achieving a high yield of protein of interest in a host cell by providing an expression construct in which the nucleic acid encoding a protein of interest is operably fused to T7 leader peptide and an expression tag in the N-terminus.

In an embodiment, the expression cassette comprises a nucleic acid encoding a protein of interest.

In an important embodiment, the expression cassette can also encode a fusion polypeptide comprising of T7 leader peptide, an expression tag and a cleavable linker fused to the N-terminal of a protein of interest.

In an embodiment, the expression cassette can also encode a fusion polypeptide comprising of T7 leader peptide, a polyhistidine tag, an expression tag and a cleavable linker fused to the N-terminal of a protein of interest.

The protein of interest is preferably a bioactive polypeptide. More preferably it includes therapeutic proteins that are useful to treat a disease in human or animals.

In an embodiment of the present invention, the expression level of the protein of interest increases by at least 85%.

In an another embodiment, the protein of interest includes therapeutic peptides which are less than 100 amino acids. In a preferred embodiment the peptide of interest includes peptides such as, but not limited to, Lira-peptide, Teriparatide, Exenatide, Lixisenatide, Teduglutide, or Semaglutide.

Expression tag refers to any peptide or polypeptide that can be attached to a protein of interest and is supposed to support the solubility, stability and/or the expression of a recombinant protein of interest.

In a further embodiment, the expression cassette comprises of a nucleic acid sequence encoding an expression tag having an amino acid sequence as set forth in SEQ ID NOs: 2-10. In a preferred embodiment, the expression cassette comprises of amino acid sequence as set forth in SEQ ID NO: 2 (LP-2) or SEQ ID NO: 8 (LP-8).

In another embodiment, the nucleic acid sequence contains the preferred codons for expression in the host cell in place of rare codons, known as codon optimization. The term “codon-optimized” as used herein refers to the alteration of codons in the gene or coding regions of the nucleic acid molecules to preferred codons in reference to the host organisms.

In certain embodiments, the nucleic acids may exhibit “codon degeneracy”. “Codon degeneracy” refers to a nucleotide that can perform the same function or yield the same output as a structurally different nucleotide.

In one embodiment, the codon-optimized expression tags comprises the nucleotide sequences as set forth in SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, and SEQ ID NO: 34.

In one embodiment, the codon-optimized expression cassettes comprises the nucleic acid encoding the expression tags, the HIS tags, the TEV recognition sites and the nucleic acid encoding the lira-peptide. The codon-optimized expression cassettes comprises the nucleotide sequences as set forth in SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 46.

In an embodiment, the expression cassette comprises a nucleotide encoding a cleavable linker peptide. Preferably the expression cassette encodes a cleavable linker peptide that is cleavable with a serine protease, an aspartic protease, a cysteine protease, or a metalloprotease.

In a preferred embodiment, the expression cassette encodes a modified TEV protease cleavage site having the amino acid sequence as set forth in SEQ ID NO: 11.

In an embodiment, the present invention provides an expression cassette for high level expression of a protein of interest comprising of the following operably linked nucleic acid sequence:
a) polynucleotide encoding T7 leader polypeptide comprising the amino acid sequence of SEQ ID NO: 1;
b) a polynucleotide encoding expression tag polypeptide comprising of an amino acid sequence selected from group comprising of SEQ ID NOs: 2-10;
c) a polynucleotide encoding a cleavable peptide linker; and
d) a polynucleotide encoding the protein of interest,
   wherein the said polynucleotide sequences of the expression cassette are operably linked to a promoter.

In another embodiment, the present invention provides an expression cassette for expression of lira-peptide, comprising of the following operably linked nucleic acid sequence:
a) a polynucleotide encoding T7 leader polypeptide comprising the amino acid sequence of SEQ ID NO: 1;
b) a polynucleotide encoding expression tag polypeptide comprising of an amino acid sequence selected from group comprising of SEQ ID NOs: 2-10;
c) a polynucleotide encoding a cleavable linker; and
d) a polynucleotide encoding lira-peptide comprising the amino acid sequence as set forth in SEQ ID No: 12 or functional variant thereof.

The expression cassette of the invention includes a promoter. The promoter could be a constitutive promoter or an inducible promoter. Constitutive or inducible promoters known to a person skilled in the art can be used in the expression cassettes in one or more embodiments of this invention.

In an embodiment, the invention provides an expression vector for expressing the protein of interest, wherein the expression vector comprises at least one copy of the above-described expression cassette.

The expression vector can further include regulatory sequences to regulate the expression of the expression cassette, transcription termination sequence, selectable markers, and multiple cloning sites. The vector can also additionally include a signal sequence for directed transport of the encoded polypeptide.

In an embodiment, the vectors suitable for the present invention include but not limited to, pD451.SR, pD431.SR, pET28, pET36, pGEX, pBAD, pQE9, pRSET and the like.

In an embodiment, the invention provides a recombinant host comprising the above-described expression vector. Suitable host cells include, but not limited to, E. coli, Corynebacterium glutamicum and Bacillus subtilis. In a preferred embodiment, E. coli is used as the recombinant host.

In an embodiment, the recombinant host cell is E. coli, which includes the strains selected from BL21 (DE3), BL21 Al, HMS174 (DE3), DH5ct, W31 10, B834, origami, Rosetta, NovaBlue (DE3), Lemo21 (DE3), T7, ER2566 and C43 (DE3).

In an embodiment, the expression vector of the invention is expressed in a recombinant host to produce a fusion peptide.

In an embodiment, the invention provides a fusion polypeptide comprising of:
a) a T7 leader polypeptide comprising the amino acid sequence of SEQ ID NO: 1;
b) an expression tag polypeptide having an amino acid sequence selected from the group comprising of SEQ ID NOs: 2-10; and
c) a cleavable peptide linker;
   fused to the amino terminal of a protein of interest to obtain the fusion polypeptide.

In an embodiment, the invention provides a fusion polypeptide comprising of:
a) a T7 leader polypeptide comprising the amino acid sequence of SEQ ID NO: 1;
b) an expression tag polypeptide having an amino acid sequence selected from the group comprising of SEQ ID NOs: 2-10; and
c) a cleavable linker peptide;
   fused to the amino terminal of lira-peptide comprising the amino acid sequence of SEQ ID No: 12 or functional variant thereof, to obtain the fusion polypeptide.

In one embodiment, the present invention provides fusion polypeptides as set forth in SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, and SEQ ID NO: 22.

The present invention also provides a method for increased production of protein of interest, wherein the said protein of interest is obtained by cleaving the fusion protein at the cleavable linker.

In an embodiment, the present invention also provides a method for producing a protein of interest, said method comprises the steps of:
a) constructing an expression construct, wherein the expression construct comprises of:
   i. a polynucleotide encoding T7 leader polypeptide comprising the amino acid sequence of SEQ ID NO: 1;
   ii. a polynucleotide encoding expression tag polypeptide comprising of an amino acid sequence selected from group comprising of SEQ ID NOs: 2-10;
   iii. polynucleotide encoding a cleavable peptide linker; and
   iv. a polynucleotide encoding the protein of interest;
b) inserting the expression construct into an expression vector;
c) transforming a recombinant host with the expression vector;
d) growing the recombinant host under optimal conditions for expressing a fusion protein, wherein the fusion protein comprises of T7 leader polypeptide, expression tag, and cleavable peptide linker fused to N-terminal of protein of interest
   e) isolating the fusion protein from the cell; and
f) cleaving the fusion protein at the cleavable linker peptide to obtain the protein of interest.

In an embodiment, the present invention also provides a method for producing lira-peptide, said method comprises the steps of:
a) constructing an expression construct, wherein the expression construct comprises of:
   i. a polynucleotide encoding T7 leader polypeptide comprising the amino acid sequence of SEQ ID NO: 1;
   ii. a polynucleotide encoding expression tag polypeptide comprising of an amino acid sequence selected from group comprising of SEQ ID NOs: 2-10;
   iii. polynucleotide encoding a cleavable peptide linker; and
   iv. a polynucleotide encoding lira-peptide comprising the amino acid sequence of SEQ ID No: 12 or functional variant thereof;
b) inserting the expression construct into an expression vector;
c) transforming a recombinant host with the expression vector;
d) growing the recombinant host under optimal conditions for expressing a fusion protein, wherein the fusion protein comprises of T7 leader polypeptide, expression tag, and cleavable peptide linker fused to N-terminal of lira-peptide;
e) isolating the fusion protein from the cell; and
f) cleaving the fusion protein at the cleavable linker peptide to obtain the lira-peptide.

Liraglutide, an analog of human GLP-1 and acts as a GLP-1 receptor agonist. Liraglutide is made by attaching a C-16 fatty acid (palmitic acid) with a glutamic acid spacer on the remaining lysine residue at position 26 of the peptide precursor (lira-peptide as set forth in SEQ ID NO: 12).

In another embodiment, the invention provides a method for production of Lira-peptide, said method comprising the steps of:
a) Construction of a recombinant vector (expression construct),
b) Transformation of the expression construct into Escherichia coli,
c) Evaluation of the clones for peptide expression,
d) Purification of tagged Lira-peptide,
e) Cleavage of the N-terminal fusion tag and purification of Lira-peptide.

Standard recombinant DNA and molecular cloning techniques used herein are well known in the art and are described by Sambrook, J., Fritsch, E. F. and Maniatis, T., Molecular Cloning: A Laboratory Manual, Third Edition, Cold Spring Harbor Laboratory Press, Cold Spring Harbor, N.Y. (2001)

The above disclosure generally describes the present invention. A more complete understanding can be obtained by reference to the following specific examples. This example is described solely for the purposes of illustration and are not intended to limit the scope of the invention. Although specific terms have been employed herein, such terms are intended in a descriptive sense and not for purposes of limitation.

Different embodiments of the present invention are further defined by way of the following examples. The following examples are for the purpose of illustration of the invention and not intended in any way to limit the scope of the invention.

### EXAMPLES

### Example 1: Lira-Peptide Expression Plasmid Construction

The DNA encoding lira-peptide with a combination of N-terminal fusions (FIG.-A, 1B, 1C, 1D) and (Seq ID NO: 13 to 23) were codon-optimized to E. coli and synthesized.

The E. coli expression plasmid pD451.SR was procured from ATUM in a linearized form (SapI digested). The synthesized DNA of lira-peptide combined with different N-terminal fusions was digested with SapI restriction enzyme. The restriction digested fragments were ligated with the pD451. SR linear plasmid and transformed into Escherichia coli strain. The resultant plasmids containing lira-peptide expression cassettes (FIG.-2A, 2B, 2C, 2D & 2E) were confirmed by nucleotide sequencing.

The codon-optimized expression tags comprises the nucleotide sequences as set forth in SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, and SEQ ID NO: 34.

The codon-optimized expression cassettes comprises the nucleic acid encoding the expression tags, the HIS tags, TEV recognition sites and the nucleic acid encoding the lira-peptide. The codon-optimized expression cassettes comprises the nucleotide sequences as set forth in SEQ ID NO: 35, SEQ ID NO: 36, SEQ ID NO: 37, SEQ ID NO: 38, SEQ ID NO: 39, SEQ ID NO: 40,SEQ ID NO: 41, SEQ ID NO: 42, SEQ ID NO: 43, SEQ ID NO: 44, SEQ ID NO: 45, SEQ ID NO: 46 and SEQ ID NO: 47.

### Example 2: Transformation into E. coli and Peptide Expression

The sequence confirmed plasmid DNA's containing cassettes LP1 to LP11 were transformed into E. coli BL21 (DE3) by calcium chloride heat-shock transformation method, followed by plating on LB agar containing 50 μg/ml Kanamycin antibiotic. The transformed E. coli cells were cultured in 5 ml LB media containing 50 g/ml Kanamycin overnight at 37° C. in a shaker incubator, followed by dilution of the culture with new media in 1:100 ratios and allowed it grow until OD reaches ˜0.6.

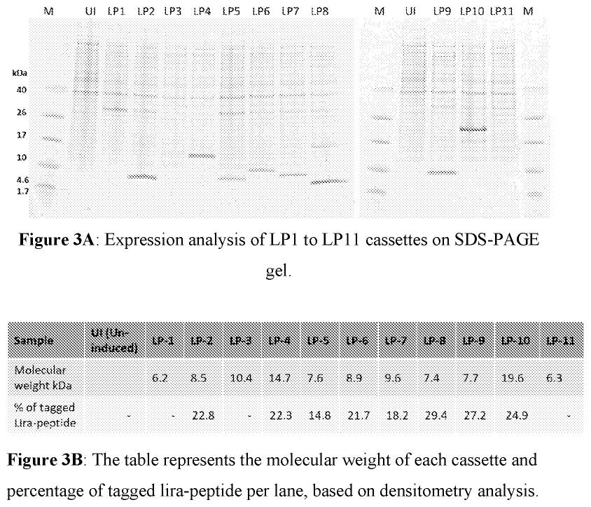

Then IPTG was added to a final concentration of 1 mM and incubated in a shaker incubator for 4 hrs at 37° C. The cultured cell OD's were normalized before loading onto SDS-PAGE gel for the peptide expression analysis (FIG. 3A). The expression of lira-peptide was observed on the gel for all the cassettes except LP1 (SEQ ID NO: 35), LP3 (SEQ ID NO: 37), and LP11 (SEQ ID

NO: 45).

The gels were subjected to densitometry analysis to quantify the lira-peptide band density among each lane's total protein, using the Image-Quant 800 gel documentation system and its software from GE.

The selection of clones was based on the smallest size of the expression tag and higher densities of lira-peptide bands on the gel so that the lira-peptide yields are expected to be higher.

It was identified that the lirapeptide without expression tag didn't show any expression on the gel, which indicates that the expression tag is essential for the expression. The LP2 and LP8 clones were selected for further analysis because their expression tag sizes were comparatively small, and the lira-peptide band densities were higher (FIG. 3B).

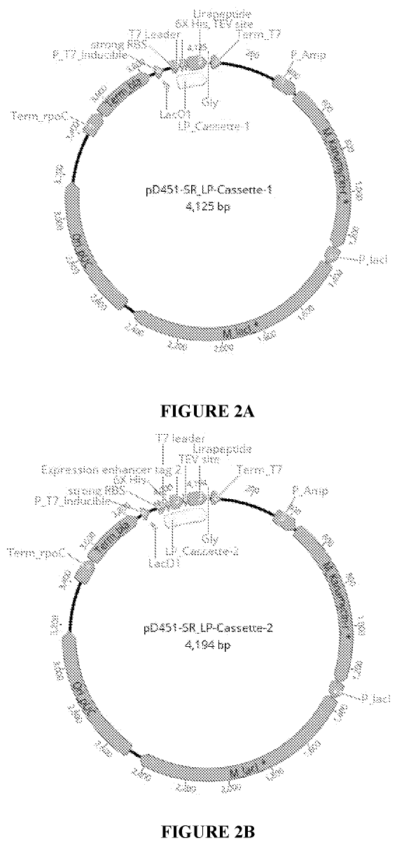

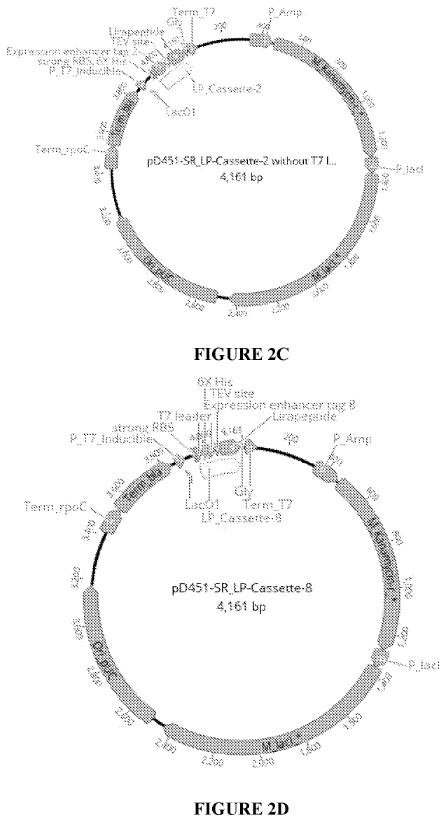

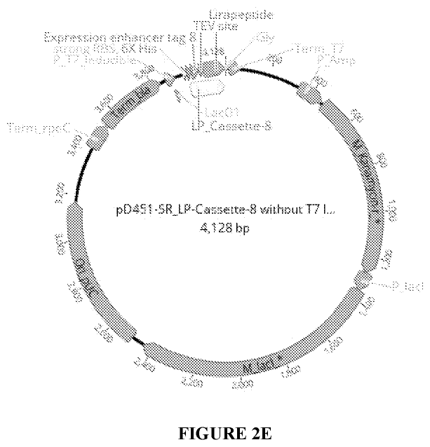

To identify whether there is a synergistic effect between T7 leader and expression tag on lira-peptide expression in LP2 and LP8 clones, we have constructed and evaluated LP2 and LP8 cassettes without T7 leader (Seq ID NO 24 & 25) and (FIG. 2C & 2E).

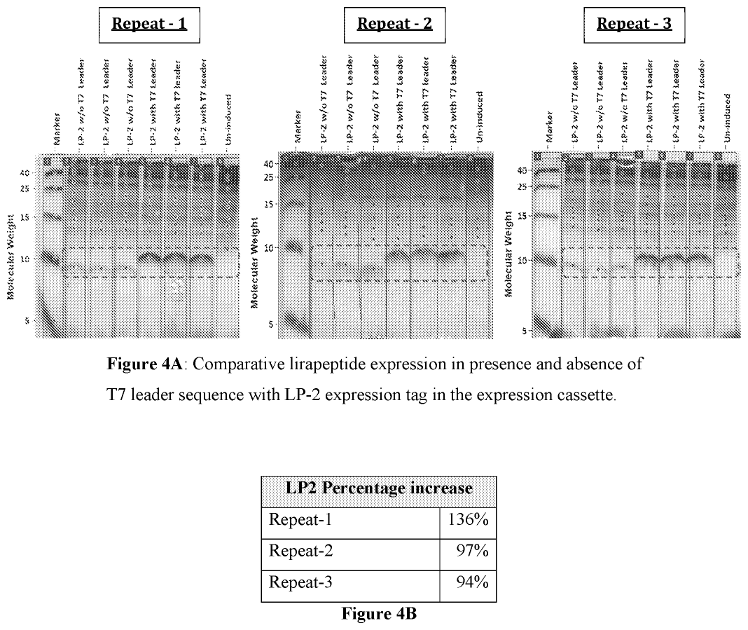

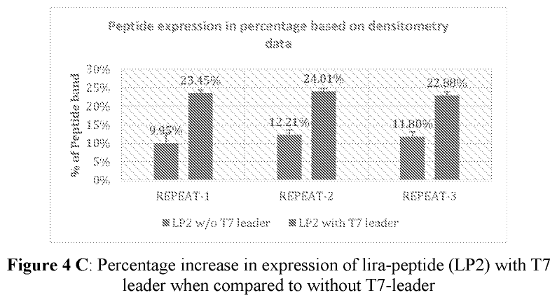

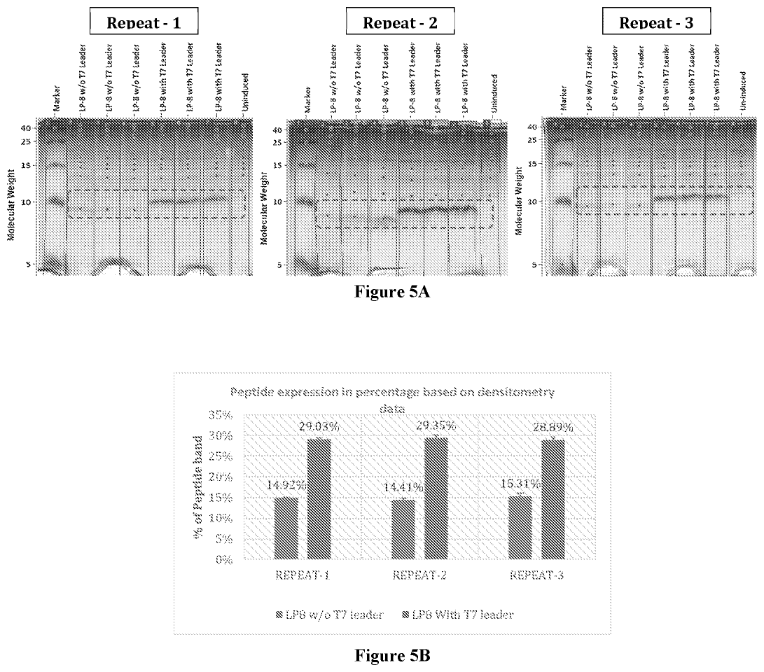

It was identified that the peptide expression of LP2 and LP8 with T7 leader was at least 85% percent higher than the LP2 & LP8 without T7 leader (FIGS. 4A, B, C & 5A, B, C).

### Example 3: Purification of Lira-Peptide containing N-terminal Fusion

The cells were lysed using sonication procedure, followed by centrifugation of lysate, and then the insoluble pellet was dissolved in 8M urea.

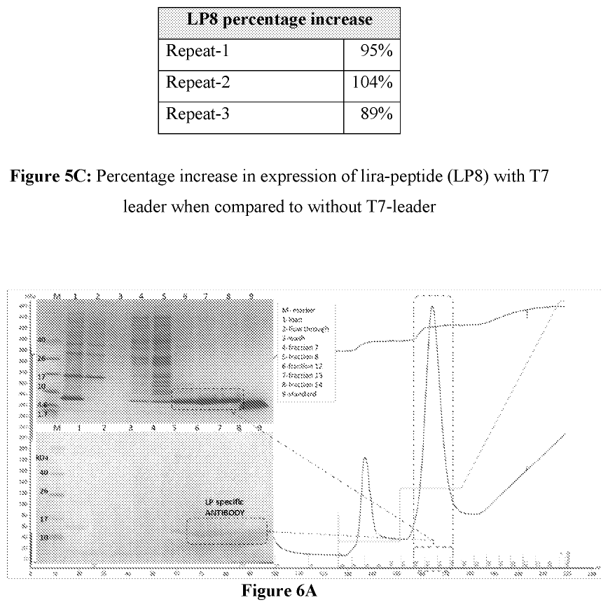

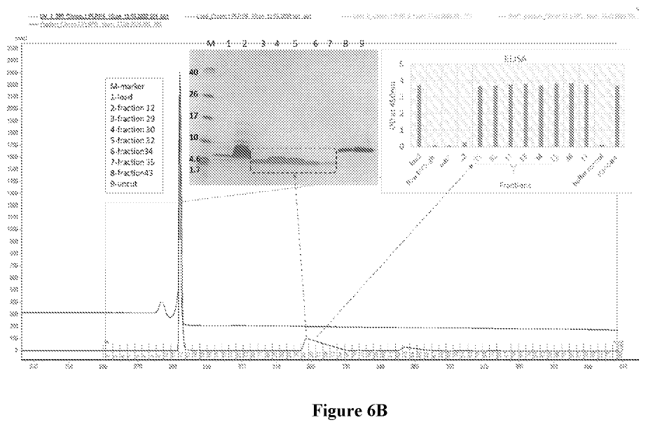

The sample was loaded onto Ni-NTA matrices; His-tagged proteins are bound, and other proteins pass through the matrix. After washing, the his-tagged peptide was eluted using imidazole with a step gradient to separate the peptide from impurities (FIG. 6A).

### Example 4: Removal of N-terminal fusion tag and purification of Lira-peptide

The purified tagged lira-peptide was subjected to TEV protease to cleave the N-terminal fusion tag. Then the sample was loaded onto reverse phase column chromatography to purify the lira-peptide (FIG. 6B). The purified lira-peptide amino acid sequence and intact mass were confirmed using LC/MS.

### Example 5: Expression of Teriparatide

The DNA encoding Teriparatide with amino acid sequence of SEQ ID NO: 49 with a combination of N-terminal fusions, comprising of T7 Leader peptide, polyhistidine tag, expression tags (SEQ ID NOs: 26-34), and modified TEV cleavable linker were codon-optimized to E. coli and synthesized. The expression constructs comprising the expression tags of SEQ ID Nos: 26-34 are termed as TP2-TP10. The expression construct TP1 does not contain any expression tag, and the expression construct TP11 comprises of T7 leader+6XArg +TEVrecognition site+Teriparatide.

The E. coli expression plasmid pD451.SR was procured from ATUM in a linearized form (SapI digested). The synthesized DNA of Teriparatide combined with different N-terminal fusions was digested with SapI restriction enzyme. The restriction digested fragments were ligated with the pD451.SR linear plasmid and transformed into Escherichia coli strain. The resultant plasmids containing Teriparatide expression cassettes were confirmed by nucleotide sequencing.

The sequence confirmed plasmid DNA's containing cassettes TP1 to TP11 were transformed into E. coli BL21 (DE3) by calcium chloride heat-shock transformation method, followed by plating on LB agar containing 50 μg/ml Kanamycin antibiotic. The transformed E. coli cells were cultured in 5 ml LB media containing 50 μg/ml Kanamycin overnight at 37° C. in a shaker incubator, followed by dilution of the culture with new media in 1:100 ratios and allowed it grow until OD reaches ˜0.6.

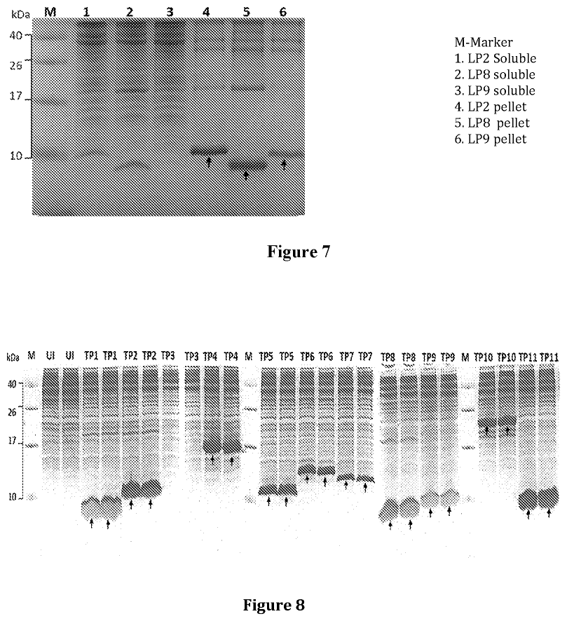

Then IPTG was added to a final concentration of 1 mM and incubated in a shaker incubator for 4 hrs at 37° C. The cultured cell OD's were normalized before loading onto SDS-PAGE gel for the peptide expression analysis (FIG. 8). The uninduced (UI) sample was used as control. The expression of Teriparatide was observed on the gel for all the cassettes, except for TP3.

### ADVANTAGES OF THE INVENTION

In the present study, high expression levels of lira-peptide were achieved using very short fusion tags such as tag LP-2 (23AA) and tag LP-8 (12AA) combined with T7 leader. The fusion tag would induce aggregation into inclusion bodies, increase the stability of protein, protect the peptides from host cell degradative enzymes' action and also help in post expression purification. FIG. 7, which shows expression of lira-peptide in soluble and insoluble fractions, indicates that majority of the fusion-peptide was identified in insoluble fraction. The tags of the present invention are very small in size when compared to the commonly used fusion tags such as GST (26 kDa), Thioredoxin Trx (12 kDa), MBP tag (42 kDa), Ketosteroid isomerase (KSI) 14 kDa, and SUMO 14 kDa. Using the shortest possible peptide tags for improving the expression of the peptide of interest can overcome the limitations of using large fusion tags and improve the yields, thereby reducing the cost of manufacturing.

1. An expression cassette for expression of a protein of interest, wherein the said expression cassette comprises of:
a) polynucleotide encoding T7 leader polypeptide comprising the amino acid sequence of SEQ ID NO: 1;
b) a polynucleotide encoding expression tag polypeptide comprising of an amino acid sequence selected from the group comprising of SEQ ID NO: 2-10;
c) a polynucleotide encoding a cleavable peptide linker; and
d) a polynucleotide encoding the protein of interest,
wherein the said polynucleotide sequences of the expression cassette are operably linked to a promoter.

2. The expression cassette of claim 1, wherein the said expression cassette further comprises of a polynucleotide encoding a polyhistidine tag.

3. The expression cassette of claim 1, wherein the cleavable linker comprises of a modified TEV protease cleavage site having the amino acid sequence as set forth in SEQ ID NO: 11.

4. The expression cassette of claim 1, wherein the protein of interest comprises of therapeutic peptides which are less than 100 amino acids.

5. The expression cassette of claim 1, wherein the protein of interest is selected from the group comprising of Lira-peptide, Teriparatide, Exenatide, Lixisenatide, Teduglutide, and Semaglutide.

6. The expression cassette of claim 1, wherein the protein of interest is Lira-peptide.

7. The expression cassette of claim 1, wherein the expression level of the protein of interest increases by at least 85%.

8. An expression cassette for expression of lira-peptide, wherein the said expression cassette comprises of:
a) a polynucleotide encoding T7 leader polypeptide comprising the amino acid sequence of SEQ ID NO: 1;
b) a polynucleotide encoding expression tag polypeptide comprising of an amino acid sequence selected from the group comprising of SEQ ID NO: 2-10;
c) a polynucleotide encoding a cleavable peptide linker; and
d) a polynucleotide encoding lira-peptide comprising the amino acid sequence as set forth in SEQ ID NO: 12 or a functional variant thereof,
wherein the said polynucleotide sequences of the expression cassette are operably linked to a promoter.

9. The expression cassette of claim 8, wherein the cleavable linker comprises of a modified TEV protease cleavage site having the amino acid sequence as set forth in SEQ ID NO: 11.

10. The expression cassette of any one of claims 1-8, wherein the said expression cassette comprises of polynucleotide sequence as set forth in SEQ ID NOs: 36-44

11. An expression vector for expression of a protein of interest, wherein the said expression vector comprises of at least one copy of expression cassette from any one of claims 1-10.

12. The expression cassette of claim 1 or the expression vector of claim 11 for use in the expression of a protein of interest.

13. A host cell for enhanced production of a protein of interest comprising of an expression vector, wherein the said expression vector comprises of any one of expression cassettes from claims 1-10.

14. The host cell of claim 13, wherein the said host cell is selected from a group comprising of E. coli, Corynebacterium glutamicum and Bacillus subtilis.

15. The host cell of claim 14, wherein the E. coli strain is selected from the group comprising of BL21 (DE3), BL21 Al, HMS174 (DE3), DH5ct, W31 10, B834, origami, Rosetta, NovaBlue (DE3), Lemo21 (DE3), T7, ER2566 and C43 (DE3).

16. A fusion polypeptide comprising of:
a) a T7 leader polypeptide comprising the amino acid sequence of SEQ ID NO:1;
b) an expression tag polypeptide having an amino acid sequence selected from the group comprising of SEQ ID NOs: 2-10; and
c) a cleavable peptide linker;
fused to the amino-terminal of a protein of interest to obtain the fusion polypeptide.

17. The fusion polypeptide of claim 16, wherein the said fusion polypeptide further comprises of a polyhistidine tag.

18. The fusion polypeptide of claim 16, wherein the cleavable linker comprises of modified TEV protease cleavage site having the amino acid sequence as set forth in SEQ ID NO: 11.

19. The fusion polypeptide of claim 16, wherein the protein of interest comprises of therapeutic peptides which are less than 100 amino acids long.

20. The fusion polypeptide of claim 16 wherein the protein of interest is selected from the group comprising of Lira-peptide, Teriparatide, Exenatide, Lixisenatide, Teduglutide, and Semaglutide.

21. The fusion polypeptide of claim 16, wherein the protein of interest is lira-peptide as set forth in amino acid sequence of SEQ ID NO: 12 or functional equivalents thereof.

22. The fusion polypeptide of claim 16, wherein the said fusion polypeptide comprises of an amino acid sequence as set forth in SEQ ID NOs: 14-22.

23. A method of producing a protein of interest, wherein the said method comprises the steps of:
a) culturing the host cell of any one of claims 13-15, under favorable conditions, to obtain the fusion polypeptide of any one of claims 16-22;
b) isolating the fusion polypeptide obtained from step a); and
c) cleaving the fusion polypeptide obtained from step b) at the cleavable linker to obtain the protein of interest.

24. The method of claim 23, wherein the protein of interest is selected from the group comprising of Lira-peptide, Teriparatide, Exenatide, Lixisenatide, Teduglutide, and Semaglutide.

25. The method of claim 23, wherein the protein of interest is lira-peptide as set forth in amino acid sequence of SEQ ID NO: 12 or functional equivalents thereof.
